(19) United States
(12) Patent Application Publication
(10) Pub. No.: US 2014/0249646 A1
(43) Pub. Date: Sep. 4, 2014

(54) SYSTEM FOR RECORDING ELECTRONEUROGRAPHIC ACTIVITY

(71) Applicants: Clemens Florian Eder, London (GB); Mads Peter Andersen, Hjorring (DK)

(72) Inventors: Clemens Florian Eder, London (GB); Mads Peter Andersen, Hjorring (DK)

(73) Assignee: NEURODAN A/S, Aalborg SV (DK)

(21) Appl. No.: 14/241,970

(22) PCT Filed: Aug. 27, 2012
(86) PCT No.: PCT/DK2012/050311

§ 371 Date: Apr. 25, 2014

(30) Foreign Application Priority Data
Aug. 29, 2011 (DK) PA 2011 0646

(51) Int. Cl. A61B 5/04; A61F 2/72; A61N 1/36; A61B 5/00

(52) U.S. Cl. CPC A61B 5/04001; A61B 5/725; A61F 2/72; A61B 5/4836; A61N 1/36007; A61N 1/36003
USPC 623/25; 600/377; 607/41; 607/49

(57) ABSTRACT

A system for recording electroneurographic activity comprising at least three electrodes capable of sensing a nerve signal from a peripheral nerve and means for receiving and processing the sensed nerve signal to identify a signal indicative of a specific action being a movement of a body part performed by the patient and for producing a control signal in response thereto featuring means for rejection of signals originating from biological interference sources without adversely affecting the electroneurographic activity being measured.

### TECHNICAL FIELD

The present invention is generally concerned with the art of sensing neural signals from nerves. In particular it relates to amplification and filtering of nerve activity in order to determine the best timing for initiating electrical stimulation of nerves, or the control of a prosthesis.

### BACKGROUND OF THE INVENTION

Electrical stimulation of nerve trunks and their branches is known to be effective in the treatment of a variety of neurological disorders in humans, spanning from treatment of incontinence to gait disorders. Sensing and recording nerve signals is a discipline that aims for obtaining valuable input for actively controlling the timing of the electrical stimulation of nerves. The recorded nerve signals can also be used for controlling equipment placed outside the body as e.g. prostheses that serve as functional replacement of body parts.

When it comes to the art of electrical stimulation of nerves for the treatment of gait disorders, especially for correcting drop-foot, electrodes are placed in the proximity of the peroneal nerve or its branches. An implantable pulse generator connected to the electrode arrangement generates a pattern of pulses to stimulate the nerve which will cause the foot dorsiflexor muscles to contract. Thus the foot will be lifted and it will be possible for the patient to swing the leg more naturally while walking. An example of a system for correction of drop-foot is known from EP 1 257 318 B1 to Neurodan A/S. The document covers the medical aspects and discloses examples of various preferred embodiments. For the triggering of the electrical stimulation of the nerve, according to the wanted reaction of the foot, the use of a heel switch is disclosed. The heel switch can be either connected to the pulse generator with electrical wires or it can include a wireless transmitter module for triggering the pulse generator. For the interface between the pulse generator and the electrodes the system comprises an inductive link, an antenna to be mounted on the skin of the patient and a counterpart in form of an implantable antenna adapted to be implanted in the thigh of the patient. In a further embodiment it is shown that neural information recorded on e.g. the Sural nerve can be used for determining certain gait events such as heel strike and heel lift. For detecting the neural information a nerve recording electrode arrangement is used, where the electrodes—in the preferred embodiment—are being placed inside the wall of an insulating and elastic silicone rubber tube, representing a CUFF being wrapped around the nerve. The CUFF is used in one embodiment for a multipolar nerve stimulation and recording electrode arrangement, where the electrodes are switched between a mode of recording nerve signals and a mode where electrical nerve stimulation is carried out. As can be seen in FIG. 1, natural sensors can be used as trigger input for a drop foot stimulator. Gait related information can be either sensed from a dedicated sensing electrode arrangement on a purely sensory nerve, or through the same set of electrodes that the mixed common peroneal nerve (sensory and motor branches) is being stimulated with.

When it comes to recording information from natural sensors in living beings, information is encoded as action potentials. These are propagating along nerve fibers, either from their natural sensors, or to their muscles. An action potential is a transient change in the voltage between the intracellular (within the nerve fiber) and extracellular space (outside the nerve fiber) on either side of the membrane, as result of a mechanical, electrical or chemical stimulus that changes the electrochemical balance. This local disturbance can cause imbalance in the neighboring nerve tissue, allowing the action potential to propagate along the nerve fiber. As a result of the short lasting disturbance at any given point on the nerve fiber, ionic currents are flowing into and out of the membrane of the nerve cells. It is these membrane action currents, which allow the pickup of nerve activity with electrodes adjacent to the nerve, so-called extracellular electrodes.

If an electrode is placed on a cut nerve ending where the intracellular fluid makes good contact with restricted extracellular fluid, and a second electrode is placed further along the uninjured nerve, the shape of the extracellularly recorded action potential is identical to that of the membrane action potential at the second electrode [R. B. Stein and K. G. Pearson. amplitude and form of action potentials recorded from unmyelinated nerve fibres. J. Theoretical biology 32:539-558, 1971]. FIG. 2, shows the setup for a monopolar recording with a single electrode placed around the nerve. The reference electrode is arranged far away from the recording electrode. Whenever the action potentials propagate underneath the electrode, the associated action currents causes voltage differences that can be picked up by the extracellular electrode. The voltage waveform approaches a scaled version of the action potential, with a scaling factor that depends on the transverse and longitudinal conductivity of the medium surrounding the nerve fibers.

The monopolar configuration has the disadvantage that other biological interference—as for instance caused by adjacent muscle activity—will be indistinguishably picked up between recording and reference electrode. This situation can be greatly improved by recording nerve activity between two adjacent electrodes with an instrumentation amplifier which can greatly reduce any common mode interference as shown in FIG. 3. If the electrodes are aligned parallel to the gradient of the electric interference field, a tiny fraction of the greatly extended biological interference field can be sampled as differential voltage, which is increasing with the inter-electrode distance. But the inter-electrode distance cannot be made arbitrary small, because the wavelength of the action potentials increases with the nerve conduction velocity, and thus requires a larger inter-electrode distance for proper spatial sampling especially for fast conducting nerve fibers.

As previously mentioned, the amplitude of the action potentials recorded with extracellular electrodes is also dependent on the conductivity of the surrounding medium. It was found that the amplitude was proportional to the ratio between extracellular and axioplasmatic (i.e. the ohm'ic resistance inside of the nerve) resistivity [A. L. Hodgkin and W. A. Rushton. The electrical constants of a crustacean nerve fibre. Proc. R. Soc. Med. 134 (873):444-479, 1946].

Researchers have shown that if a nerve is brought into another electrically isolating medium like air (lifted the nerve with the attached hook electrode from the biological medium) or paraffin, the voltages significantly increase [L. Hermann. Untersuchungen ueber die Aktionsstroeme des Nerven: Teil I I. Pfluger's Arch. ges. Physiol. 24:246-294, 1881],[K. S. Cole and H. J. Curtis. Membrane Potential of the Squid Giant Axon during current flow. J. Gen. Physiol. 24 (4):551-563, 1941]. This led researchers to the idea of surrounding the recording electrodes by an insulating silastic nerve cuff [R. B. Stein, D. Charles, L. Davis, J. Jhamandas, A. Mannard, and T. R. Nichols. Principles underlying new methods for chronic neural recording. Canadian Journal of Neurological Sciences: 235-244, 1975],[J. A. Hoffer and G. E. Loeb. Implantable electrical and mechanical interfaces with nerve and muscle. Ann. Biomed. Eng 8:351-369, 1980]. These cuff electrode arrangements can be produced by molding the electrodes into silastic sheets that are wrapped around the nerve, and closed by a suture. As the silicone cuff is surrounding the recording electrodes, it also reduces the picked up differential interference voltages.

The differential interference can be further reduced by connecting three amplifiers in a double-differential configuration as shown in FIG. 4. This scheme was first introduced by Pflaum et al. [Pflaum et al. An improved nerve cuff recording configuration for FES feedback control system that utilizes natural sensors. Proc. IFESS, pp. 407-410,1995] for electroneurographic measurements and was referred to as “true-tripolar” configuration. The principle is based on the fact that interference currents cause instantaneous—and ideally equal—voltage differences that are present in each adjacent electrode pair. Thus, Vt1 and Vt2 are of equal phase (FIG. 4). An additional amplifier can be used to nullify the interference by subtracting Vt1 from Vt2. Even if the amplitudes are not equal, for instance due to the difference in inter-electrode impedances Rt1 and Rt2, the interference can be theoretically nullified by proper adjustment of the gain ratio between G1 and G2.

On the other hand, the superpositions of a great number of action potentials that propagate along the longitudinal neural axis constitute the signal of interest. Their conduction velocity, reaching up to approximately 100 m/s, causes a delay between each bipolar recording of amplifier G1 and G2. If the inter-electrode spacing is sufficient for a given nerve conduction velocity, the phase differences will be large enough to prevent the double differential amplifier from nullifying the nerve signals as well. Under the right conditions (i.e. conduction velocity and inter-electrode spacing), the action potential's peak reaches the center electrode, while the end electrodes are located at the very beginning or the very end of the action potential wave. One amplifier detects the positive rising phase, while the other detects the falling phase. Thus, the double differential amplifier configuration allows the amplification of the desired out-of phase nerve activity, while greatly reducing the instantaneous bioelectric interference.

However, the interference reduction performance might be subject to change if the ratio between gains G1 and G2 is fixed. Changes in the impedance balance between Rt1 and Rt2, as well as non-linear field effects that depend on the location of the interference source [Triantis I. F. & Demosthenous A. The effect of interference proximity on cuff imbalance. February 2006, IEEE Trans. BME, 53(2), p. 354-7] might require a re-adjustment of the gains G1 and G2 to maintain the desired interference rejection. The need for an adaptive system that automatically tunes gains G1 and G2 was addressed in [Demosthenous A. et al. Design of an adaptive interference reduction system for nerve cuff electrode recording. April 2004. IEEE Trans. Circuits & Systems 51(4), p. 629-639].

The above described research overview points out the basic principle behind recording nervous activity and points out methods for the rejection of undesired bioelectric artifacts, like those being attributed to muscular activity. The methods are based on arithmetic operations on signals from pairs of electrodes that are carried out by hardware, before sampling and converting the signal into the digital domain.

However, even though the above mentioned state of art presents a solution to the problem of bioelectrical interference, the solution has the drawback that changes in the impedance balance requires adaptability of the gain, but doing so by a closed loop can lead to low performance or instability, which renders the system inadequate for providing reliable input to a system relying on events encoded in nerve signals. Thus a more stable and reliable solution is desired.

### DESCRIPTION OF THE INVENTION

It is an object of the present invention to provide a system for recording electroneurographic activity for recognizing specific patterns in the recorded nerve signal despite the presence of bioelectric interference.

This is according to the invention achieved by providing a system for recording neural activity comprising at least three electrodes that are adapted to be arranged along the longitudinal axis of a peripheral nerve and further includes means for amplifying and processing the sensed nerve activity where the system includes a digital adaptive filter configured to reject bioelectric interference sources with overlapping frequency spectra.

A clear separation between the neural signal of interest and the bioelectric interference cannot be easily achieved by standard filtering techniques. The frequency distribution of the neural signal of interest highly depends on the chosen recording configuration (distance and number between electrodes, nerve conduction velocity, etc).

An adaptive filter can be applied in circumstances in which the primary signal (containing the signal of interest) is obstructed by interference, whose characteristics can be derived by an independent set of additional sensors. Once an independent model of the interference can be obtained such that it is uncorrelated to the signal of interest, it can be applied to an adaptive filter before subtracting the filter output from the primary signal. The filter has to be iteratively adapted such that the error, that is the difference between the primary signal and the interference model, is minimal. This error constitutes the signal of interest at the output of the filter. If a perfect model of the interference source can be found, and if it is statistically independent from the signal of interest, it can be shown that the error itself constitutes the signal of interest.

For providing a solution to the outlined problem, the present invention addresses the problem of finding a reference signal (the model of the muscular interference) from the same set of sensors (electrodes) that are being used for measuring the signal of interest. This can be achieved by taking the different cross correlation behaviour between signal of interest (the nerve signal) and the bioelectric interference into account. In the present invention a reference signal is being derived from the measurements of multiple electrodes, such that the reference signal is proportional to the bioelectrical interference.

The invention is conceived for at least three electrodes that are arranged along the longitudinal nerve axis. The electrodes are typically extracellular electrodes that are either placed circumferentially around the nerve, or which are placed in-between or even within the individual nerve fascicles. Neither the individual properties of the electrodes, nor the type of their fixation is of relevance for the present invention.

The interference is instantaneously present on all electrodes, and is therefore positively correlated across the individual bipolar channels. The nerve signals are however negatively correlated, since two adjacent bipolar channels are presented with a rising and a falling phase of the same action potential. This makes it possible to create a model of the interference by adding two bipolar channels together, therefore increasing the interferential component while nullifying the—ideally equal—neural component. The interference model is thus independent from the signal and can be applied to the adaptive filter. The reference signal can therefore be derived by adding the signals from two or more bipolar channels.

In another aspect, the interference can be derived from the difference of two channels, where one channel is delayed. Hereby, a reference signal is derived by subtracting one bipolar channel from another, where the signal from the first bipolar channel that the neural signal passes is being delayed by the amount of time the neural signal needs to pass the inter-electrode distance.

In the preferred embodiment three electrodes that constitute two bipolar channels of a cuff electrode arrangement are employed to measure neural activity. In some situations the means for interference rejection do not preserve the original raw data, by applying irreversible arithmetic operations such as subtraction or summation. This effectively reduces the information content of the signal, for instance the direction of propagation. In the present invention the adaptive filter is configured to reject bioelectric interference while providing the raw data in more than one recorded channel.

The nerve signals are originally negatively correlated, but they become positively correlated when the first channel is being subjected to a delay which amounts to the time it takes the signal to pass from one electrode to the next, that means the time it takes to cover the inter-electrode distance. These signals are, apart from uncorrelated noise sources such as thermal noise, identical. Thus, subtracting one channel from another nullifies the signal of interest, but not the interference. This is because—due to the delay—the interference became subjected to a phase shift, and its difference is therefore non-zero. It is this difference that can act as a model for the interference, since it is uncorrelated to the nullified nerve signal. However, the problem may be ill-conditioned if this phase is very small (due to small ΔT) and if the amplitudes are almost equal. Interpreting a three-electrodes arrangement as a voltage divider with constant k=Zt1/(Zt1+Zt2), the adaptive filter has to estimate a delayed sum from a delayed difference, therefore implementing the following transfer function:

$$H ( z ) = \frac{1 + ( 1 / k - 1 ) {z}^{- 1}}{1 - ( 1 / k - 1 ) {z}^{- 1}} = \frac{1 + a ( k ) {z}^{- 1}}{1 - a ( k ) {z}^{- 1}} \qquad ( Equ . 1 )$$

Where the shift operator z⁻¹ denotes a delay of unit ΔT. The transfer function constitutes a indefinite response filter (IRR) with a zero at a(k)=z. The zero is approached for k=0.5, when the impedances Zt1 and Zt2 are exactly matching. For Zt2>Zt1 (k<0.5), the zero is outside the unit circle and instability occurs. The stability problems can be solved by approximating Equ. 1 with a FIR filter of sufficient order (to approximate the impulse response by a sequence that is bounded in value and time). The person skilled in the art will be familiar with that the longer the impulse response of the IIR filter, the more weights are necessary for the approximation through an FIR filter. This approach makes the implementation of the adaptive filter inherently stable. The adaptive filter is therefore implemented or configured as a finite-impulse response (FIR) filter with sufficient number of adaptive weights, alleviating problems of stability for ill-conditioned problem.

For all embodiments, the described multipolar electrode arrangement or the entire system may be adapted to be implanted in the human or animal body.

The system may give input to any system that aims to react on nerve signals. Especially appreciated will the system be used for giving input to a system for correcting gait related deceases as e.g. drop-foot or to a system for the control of prostheses substituting functional body parts such as artificial legs or arms.

The system can in a further embodiment be adapted to be used for giving input to a system for the treatment of incontinence.

For all embodiments, the described electrode arrangement or the entire system may be adapted to be implanted in the human or animal body. However it might also be adapted to be arranged outside the human or animal body.

### DESCRIPTION OF THE DRAWING

FIG. 1, shows an illustration of a leg region of a patient with dedicated electrodes implanted for recording nerves signals from the sural nerve, a purely sensory nerve. It also illustrates the placement of a cuff electrode placed on the peroneal nerve, for combined stimulation and sensing,

FIG. 2, shows a simplified illustration of a nerve for explanation of the problem of biological interference in monopolar recordings,

FIG. 3, shows a simplified illustration of a nerve for explanation of the problem of both common-mode and differential-mode interference voltages at the input of an instrumentation amplifier,

FIG. 4, shows a simplified illustration of a single-channel cuff electrode placed around the nerve, being subjected to an electric interference field, which can be greatly reduced by the true-tripolar configuration as shown,

FIG. 5, shows the implementation of an adaptive filter for interference rejection, where the interference is derived from the sum of inputs,

FIG. 6, shows the implementation of an adaptive filter for interference rejection, where the interference is derived from the delayed difference of inputs,

FIG. 7, shows the power spectra of the example signals that are presented to the adaptive filter and

FIG. 8, shows the power spectra of the (dotted) primary signal as input to the adaptive filter implemented by an RLS algorithm. The solid line indicates the filter output.

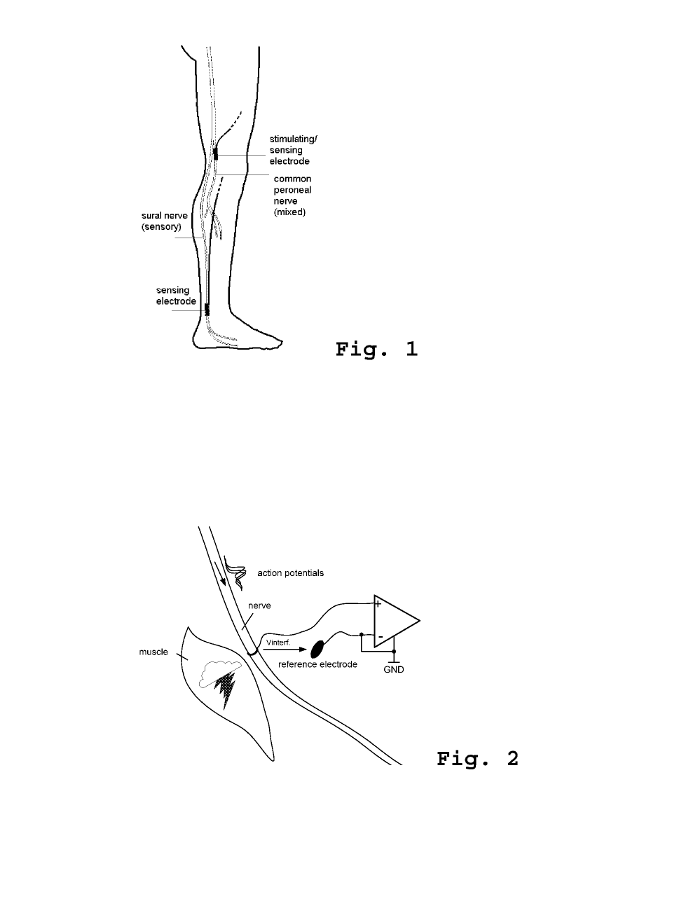

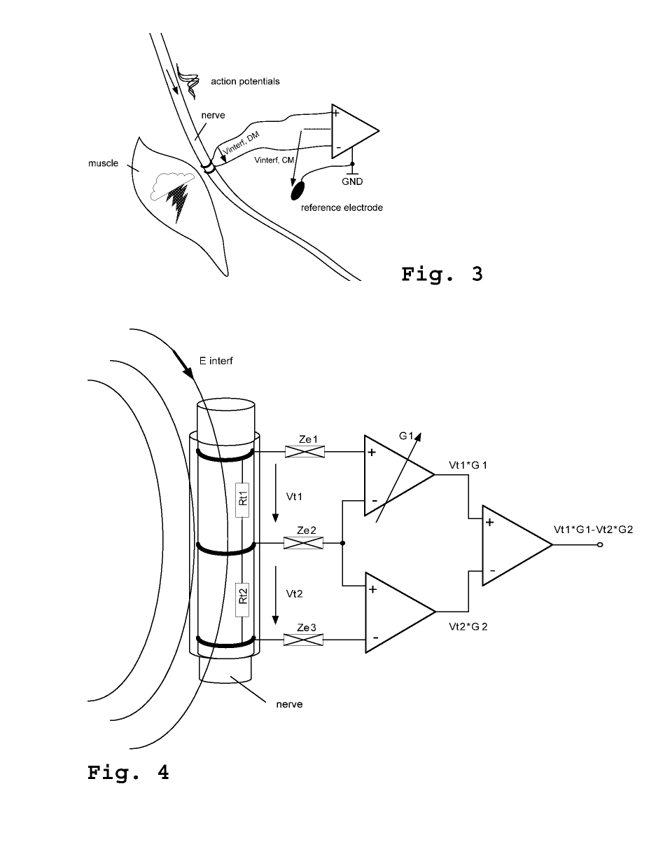

A first number of embodiments, not forming part of the invention but being useful for the understanding of the invention, has already been explained with reference to FIGS. 1 to 4 in the preamble of this application.

In a preferred embodiment the system comprises at least three equally spaced electrodes that are arranged along the longitudinal axis of the concerned nerve. The electrodes are typically extracellular electrodes that are arranged circumferentially around the nerve, or which are arranged in-between or even within the individual nerve fascicles.

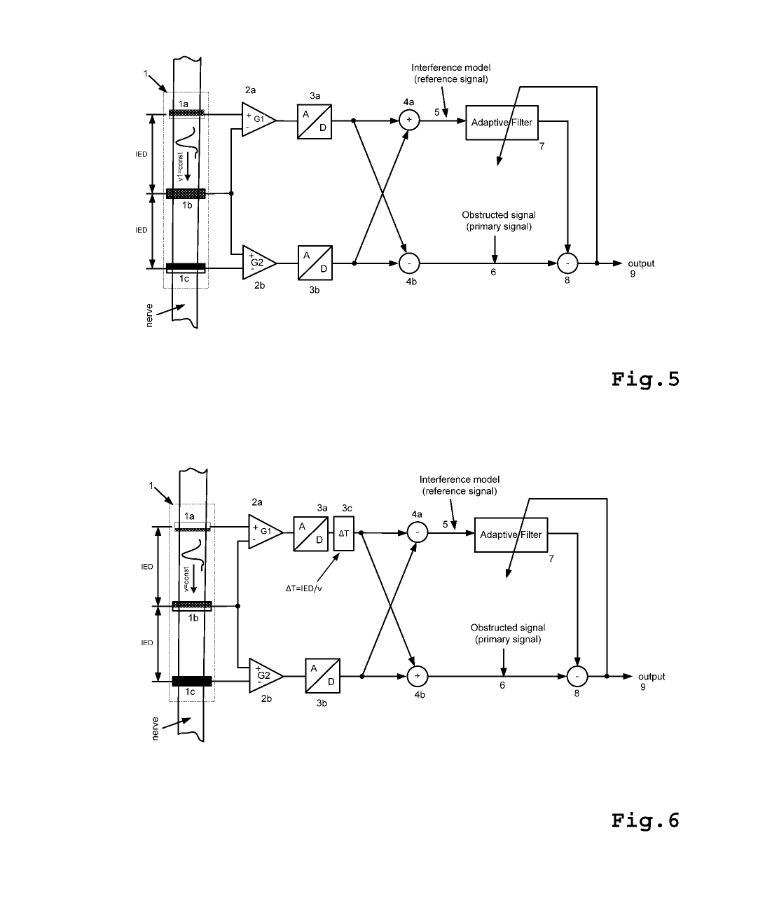

In the embodiment shown in FIG. 5, a cuff electrode arrangement is placed on a peripheral nerve and the shown electrode triplet consists of the electrodes 1a, 1b and 1c. If we assume that electrode 1a is closer to the spinal cord than electrode 1c, it will mean that action potentials traveling in the direction from electrode 1a to electrode 1c are ‘efferent’ (motor commands), and action potentials traveling the opposite directions are ‘afferent’ (sensory signals). The electrodes are spaced by the inter-electrode distance IED, with the consequence that the same waveform of the efferent action potential appears at the channel G2, with a delay corresponding to the propagation velocity v1 of the action potential arriving from the spinal cord, ΔT=IED/v1. After amplification by low-noise amplifiers 2a and 2b, the signals are digitized by the analog-to-digital converters 3a and 3b.

In this embodiment, the reference signal is obtained by summation 4a of both signals, since the interference is positively correlated among both channels. The signal of interest is subtracted 4c, as it is negatively correlated for proper inter-electrode distance and nerve propagation velocity. This primary signal thus consists of the signal of interest and residual interference, and represents one input to the filter 6.

The summed signal 5 represents a model of the interference, since the interference is positively correlated between the channels. The weights in the adaptive filter are adjusted until the difference between the primary signal and the filtered interference signal is minimal. This difference is the signal of interest.

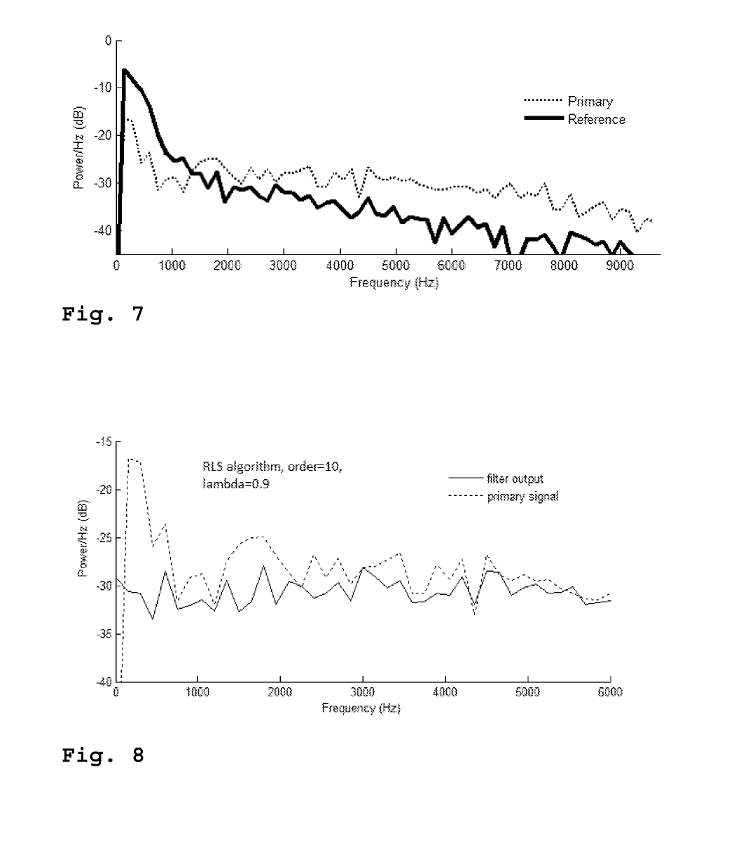

In another embodiment shown in FIG. 7, the elements 1a-3b are identical to those described in FIG. 6. In this figure it is also assumed that the nerve action potentials first pass electrode 1a and moving into direction 1c. The signals from the electrode pair 1a, 1b is being delayed by the time ΔT=IED/v1, so that both outputs of 3c and 3b are in-phase. The primary signal is obtained by summation 4b, where the interference is obtained by subtraction 4a, as the signal of interest is annihilated. The interference is not annihilated, as the first channel was shifted by ΔT=IED/v1. The reference signal 5 is however much smaller than the original interference, which is still contained in the primary signal 6. The adaptive filter 7 has to provide a high gain and proper phase shift in order to approximate the interference part contained in the primary signal. As outlined in the text, the problem might become ill-conditioned if ΔT is short, and if the interference amplitudes are equal in both channels. In this case large number of weights is required by the adaptive filter.

FIG. 7 shows an example of signals that were recorded from a tripolar cuff electrode that was implanted on the pig median nerve of a walking pig. The signals were subjected to the adaptive filter according to the embodiment described in FIG. 5. The reference signal (thick solid line) is a proper model of the interference, as it has a large peak around 200 Hz, but it contains only little energy in the band with of the signal of interest (here between 1 kHz and 10 kHz). The bioelectric interference in this example origins from muscular activity and can be clearly detected as a peak around 200 Hz. The transition between interference and signal of interest is not that sharp, which makes it difficult to use filtering without reducing energy of the signal of interest. This problem can be overcome by an adaptive filter, which requires a reference signal that is representative for the interference.

The type of adaptation algorithm is not relevant to the object of this invention. The adaptive filter can be for instance implemented by a recursive least squares or a least-means squares algorithm. As an example we show the output of the filter implemented by a recursive least-squares filter (FIG. 8), where the primary signal (the input) is indicated by a dotted line, the output is indicated by the solid line. At about 200 Hz it is clearly visible that interference power in the primary signal had been reduced by about 15 dB.

1. A system for recording electroneurographic activity, comprising:
at least three electrodes adapted to be arranged along a longitudinal nerve axis of a peripheral nerve;
at least one device configured to amplify and process the sensed nerve activity and produce a control signal in response thereto;
a digital adaptive filter configured to reject bioelectric interferences with overlapping frequency spectra;
wherein a reference signal proportional to the bioelectric interferences is derived from measurements of a plurality of the at least three electrodes.

2. A system according to claim 1,
wherein the reference signal is derived by adding signals from two or more bipolar channels.

3. A system according to claim 1,
wherein the reference signal is derived by subtracting one bipolar channel from another, where a signal from a first bipolar channel that a neural signal passes is delayed by an amount of time the neural signal requires to pass an inter-electrode distance.

4. A system according to claim 1,
wherein the adaptive filter is configured to reject the bioelectric interferences while providing raw data in more than one recorded channel.

5. A system according to claim 1,
wherein the adaptive filter is configured as a finite-impulse response (FIR) filter with sufficient number of adaptive weights to alleviate problems of stability for an ill-conditioned problem.

6. A system according to claim 1,
wherein the multi-polar electrode arrangement or the entire system is adapted to be implanted in the human or animal body.

7. A system according to claim 1,
wherein the system is giving input to a system for correcting walking disabilities such as drop-foot.

8. A system according to claim 1,
wherein the system is giving input to a system for the control of prostheses substituting functional body parts such as artificial legs or arms.

9. A system according to claim 1,
wherein the system is giving input to a system for the treatment of incontinence.

10. A system according to claim 1,
wherein the system is adapted to be arranged outside a human or animal body.

11. A system according to claim 1,
wherein the system is adapted to be implanted into a human or animal body.

12. A system for recording electroneurographic activity, comprising:
at least three electrodes configured to be positioned along a longitudinal axis of a peripheral nerve;
a device configured to amplify and process nerve activity sensed in the peripheral nerve by one or more of the at least three electrodes, the device being operable to produce a control signal in response to the sensed nerve activity;
an adaptive filter configured to reject bioelectric interferences in the sensed nerve activity using overlapping frequency spectra of a reference signal, the reference signal being proportional to the bioelectric interferences and being derived from measurements of at least two of the at least three electrodes.

13. A system according to claim 12, wherein the reference signal is derived by adding signals from two or more bipolar channels.

14. A system according to claim 12, wherein the reference signal is derived by subtracting signals from a first bipolar channel from signals of a second bipolar channel, wherein a signal from the first bipolar channel that is passed by a neural signal is delayed by an amount of time required to pass the neural signal an inter-electrode distance.

15. A system according to claim 12, wherein the adaptive filter is configured to reject bioelectric interferences while providing the raw data in more than one recorded channel.

16. A system according to claim 12, wherein the adaptive filter is configured as a finite-impulse response (FIR) filter with adaptive weights configured to alleviate instability.

17. A system according to claim 12, wherein at least portions of the system are adapted to be implanted in the human or animal body.

18. A system according to claim 12, wherein the system is configured to provide input to a system for correcting walking disabilities such as drop-foot.

19. A system according to claim 12, wherein the system is configured to provide input to a system for the control of prostheses substituting functional body parts such as artificial legs or arms.

20. A system according to claim 12, wherein the system is configured to provide input to a system for the treatment of incontinence.
